(19) United States
(12) Patent Application Publication
(10) Pub. No.: US 2025/0248827 A1
(43) Pub. Date: Aug. 7, 2025

(54) MUSCLE FLEXION INDUCTION DEVICE

(71) Applicant: Howard Arciniegas, New City, NY (US)

(72) Inventor: Howard Arciniegas, New City, NY (US)

(73) Assignee: Flamgroup LLC (d/b/a Ankleblocks), New City, NY (US)

(21) Appl. No.: 18/429,573

(22) Filed: Feb. 1, 2024

(51) Int. Cl. A61F 2/70; A61F 5/01; A61H 3/00

(52) U.S. Cl. CPC A61F 2/70; A61F 5/0127; A61H 3/00; A61F 2002/701; A61H 2003/007; A61H 2201/12

(57) ABSTRACT

A muscle flexion device may comprise a polyhedron having a trapezoidal cross-section having a slanted face that is angled with respect to a support surface and a face perpendicular to the support surface, wherein the at least one slanted face is configured to induce dorsiflexion in a posterior leg of a human user positioned in the normal gait cycle, wherein the posterior leg has a posterior foot, wherein when the posterior foot is in contact with both the support surface and the at least one slanted face, and the anterior foot is positioned anteriorly of the perpendicular face.

### FIELD OF THE INVENTION

Disclosed are embodiments of the invention that relate to, among other things, devices for inducing stretches in muscles of a user, such as, for example muscles found in the leg and/or calf of the user.

### BACKGROUND

There are numerous poses that are known for producing effective stretching of leg muscles in a user, such as, for example, Gait Analysis: Normal and Pathological Function Second Edition by Jacquelin Perry MD ScD, Judith Burnfield PhD PT. Rehabilitation Techniques for Sports Medicine and Athletic Training Seventh Edition by William E Prentice PhD ATC PT FNATA. Quick Questions in Ankle Sprains: Expert Advice in Sports Medicine First Edition by Patrick O. Mckeon, PhD ATC CSCS, Erik A. Wikstrom, PhD ATC FACSM.

There are also numerous devices that have been designed for the purpose of enabling users to stretch the leg muscle, such as, for example, slant boards of the type found in U.S. Pat. No. 8,424,465 B2, ankle rocking devices of the type found in U.S. Pat. Nos. 5,135,450 and 8,529,411, and elastic bands and Styrofoam rollers.

Each of the foregoing prior art stretches and stretching devices suffer drawbacks in terms of creating the most effective stretch for a user with the least amount of material interaction required.

### SUMMARY OF THE INVENTION

An exemplary muscle flexion device may comprise a polyhedron having a trapezoidal cross-section with a slanted face that is angled with respect to a support surface and a face perpendicular to the support surface. An exemplary muscle flexion device may also have at least one contact surface on the slanted face, whereby the polyhedron is configured to induce dorsiflexion in a posterior leg of a human user when the human user is positioned in the normal gait cycle whereby a posterior foot connected to the posterior leg simultaneously touches the at least one contact surface and the support surface. Additionally, the exemplary muscle flexion device may also be configured to allow an anterior leg to touch the support surface forward of the face perpendicular to the support surface.

An exemplary muscle flexion device previously described may be configured such that the polyhedron has a unitary trapezoidal cross-section.

An exemplary muscle flexion device like any of the ones previously described may be configured such that the angle of the slanted face with respect to the support surface is 40° to 48°.

An exemplary muscle flexion device like any of the ones previously described may be configured such that the polyhedron is configured to place a center of gravity of the human user over at least one muscle in the posterior leg.

An exemplary muscle flexion device like any of the ones previously described may be configured such that

An exemplary muscle flexion device like any of the ones previously described may be configured such that the polyhedron is configured to place a center of gravity of the human user over at least one portion of the polyhedron.

An exemplary muscle flexion device like any of the ones previously described may be configured such that the polyhedron is a trapezoid.

An exemplary muscle flexion device like any of the ones previously described may be configured such that the polyhedron is a trapezoid and the angle of the slanted face with respect to the support surface is 40° to 48°.

An exemplary muscle flexion device like any of the ones previously described may be configured such that a portion of the polyhedron comprising the slanted face is removable from the rest of the device.

An exemplary muscle flexion device like any of the ones previously described may be configured such that the anterior leg does not touch the polyhedron.

An exemplary method of flexing a muscle in a posterior leg of a user positioned in a normal gait cycle may comprise the steps of: contacting a support surface and a slanted surface of a polyhedral block having a trapezoidal cross-section placed atop the support surface using a foot of the posterior leg. The exemplary method may also include a step of placing the foot of the anterior leg forward of the polyhedral block.

An exemplary muscle flexion methodology like the one previously described may also have a step of positioning a center of gravity of the user over at least one muscle group in the posterior leg.

An exemplary muscle flexion methodology like any of the ones previously described may be configured such that the foot of the posterior leg when in contact with the polyhedral block is angled at approximately 25° to 80° with respect to the support surface.

An exemplary muscle flexion methodology like any of the ones previously described may be configured such that the foot of the posterior leg when in contact with the polyhedral block is angled at approximately 35° and 60° with respect to the support surface.

An exemplary muscle flexion methodology like any of the ones previously described may be configured such that the foot of the posterior leg when in contact with the polyhedral block is angled at approximately 40° and 50° with respect to the support surface.

An exemplary muscle flexion methodology like any of the ones previously described may also have a step of positioning a center of gravity of the user forward of a line contacting at least one stretched muscle group in the posterior leg.

An exemplary muscle flexion methodology like any of the ones previously described may also have a step of coupling the slanted surface portion of the polyhedral block to a remainder portion of the polyhedral block.

An exemplary muscle flexion methodology like any of the ones previously described may also have a step involving placing the foot of the anterior leg in contact with the polyhedral block.

An exemplary polyhedral block, such as any of the ones previously described, may also be configured to induce dorsiflexion in a posterior leg of a human user upon contact with a posterior foot of the posterior leg of the same while the human user is in a Normal Gait cycle. According to this exemplary aspect of an exemplary polyhedral block, the posterior foot of the human user may be simultaneously in touch with a support surface and at least one slanted surface on the polyhedral block. In addition to or as an alternative to any of the exemplary polyhedral blocks disclosed, an exemplary polyhedral block may have at least one flat surface in contact with a support surface and at least one side surface opposite the at least one slanted surface that is configured such that the at least one side surface, the at least one flat surface, and the at least one slanted surface are surfaces of a trapezoidal cross-section.

An exemplary polyhedral block like any of the ones previously described may be configured such that the at least one slanted surface is at an angle between approximately 25° to 80°.

An exemplary polyhedral block like any of the ones previously described may also have another slanted surface disposed on the same side as the at least one side surface.

### BRIEF DESCRIPTION OF THE DRAWINGS

FIGS. 1-3 illustrate an exemplary embodiment of an ankle block insert.

FIGS. 4-5 illustrate an exemplary embodiment of an ankle block insertion base.

FIGS. 6-8 illustrate an exemplary embodiment of a combination of an ankle block insert and a ankle block insertion base.

FIGS. 9-13 illustrate an exemplary embodiment of an ankle block.

FIGS. 14-15 illustrates an exemplary use of an exemplary ankle block inserted to a ankle block insertion base by a human user in the Normal Gait Cycle.

FIGS. 16-17 illustrates an exemplary use of an exemplary ankle block by a human user in the Normal Gait Cycle.

In the drawings like characters of reference indicate corresponding parts in the different figures. The drawing figures, elements and other depictions should be understood as being interchangeable and may be combined in any like manner in accordance with the disclosures and objectives recited herein.

### DETAILED DESCRIPTION

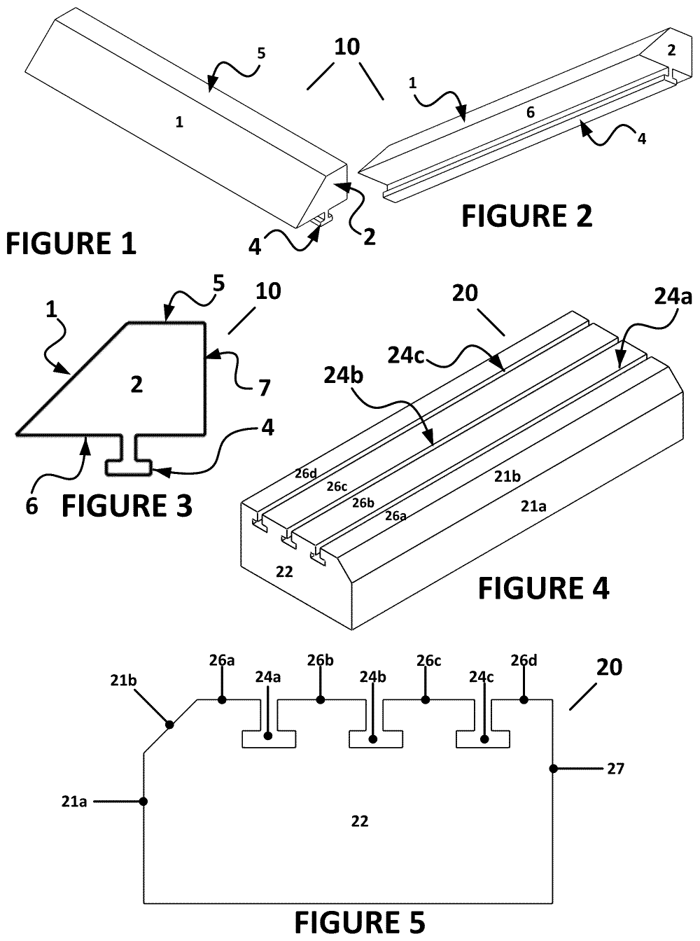

With reference to FIGS. 1-8 and 14, an exemplary slant insert 10 may be any polyhedron comprising at least one non-orthogonal face 1 that is elevated from a support surface S on which a slant base 20 rests. An exemplary slant insert 10 may have the non-orthogonal face 1 truncated by a top surface 4 and interconnected to a back face 7 by a thickness 2. In a preferred embodiment, slant insert 10 may have a trapezoidal cross-section. In another preferred embodiment, slant insert 10 may have a right angle trapezoidal cross-section. Each exemplary slant insert 10 may have a base 6 from which extends a stem 4. An exemplary stem 4 may be depicted as substantially “T” shaped, but skilled artisans may configure stem 4 in any geometry that allows it to slide into and be retained within a corresponding cavity 24a/b/c in an exemplary slant base 20, e.g., “L” shapes, “J” shapes, “F” shapes, “S” shapes, Arrow-head shapes, and ball-head shapes. In an exemplary embodiment, cavities 24a/b/c may provide for a certain amount of play or deflection with stem 4 to allow for case of use of slant insert 10. According to an exemplary embodiment, the angle of face 1 with respect to the support surface S may be between 20° to 80°, more preferably 30° to 60°, even more preferably 40° to 48°, and most preferably 42° to 44°. Additionally, an exemplary non-orthogonal face 1 may preferably be 2.0 to 4.0 inches off of the support surface S.

While an exemplary slant insert 10 and slant base 20 may be depicted as substantially solid, these structures may be porous, made from sheet metal, wood, plastic, stone, or any other structure as needed. Additionally, while any surfaces depicted for an exemplary slant base 20 may be orthogonal or straight, there is no such limitation on these features with the exception that the bottom of slant base 20 be capable of stable mounting on a support surface in accordance with the other embodiments to be described.

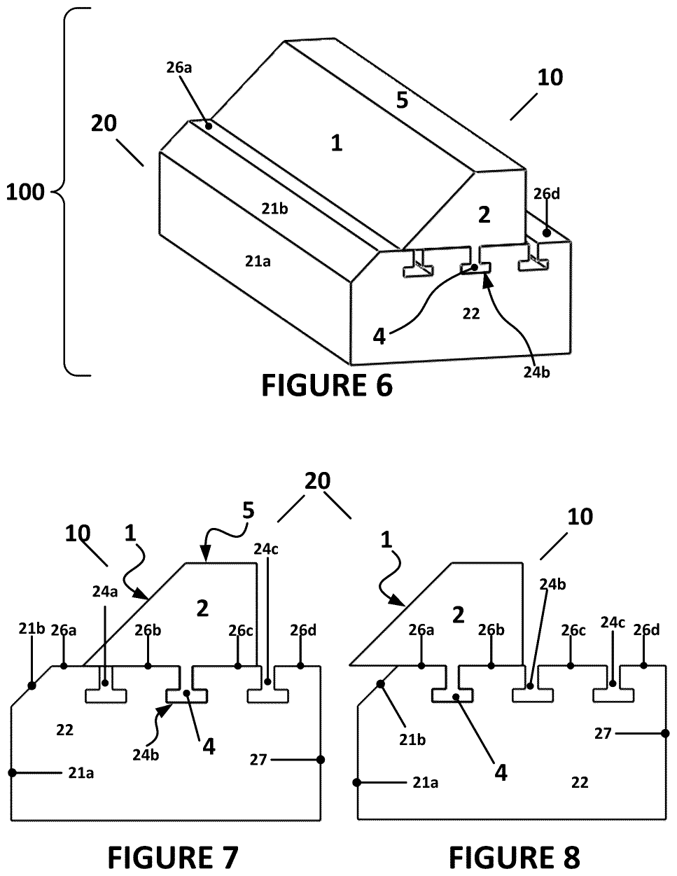

With continued reference to FIGS. 4-5, an exemplary slant base 20 may have at least one non-orthogonal face 21b juxtaposed with another face 21a, which itself may be non-orthogonal. In an exemplary embodiment, face 21a of the slant base 20 may be interconnected to a back face 27 via a thickness 22. As previously described, one or more cavities 24a/b/c may be formed in the thickness 22 of an exemplary ankle block 20 to allow for engagement by cooperatively-shaped stem 4 of exemplary slant insert 10. Interposed between each of the faces 21a/b, as the case may be, and the back face 27, may be a series of top surfaces 26a/b/c/d separating each cavity 24a/b/c from one another. The size and positions of cavities 24a/b/c may be such as to generate particular results as will be described with respect to FIGS. 6-8 and 14.

Referring to FIGS. 6-8, an exemplary ankle block system 100 may be formed from a combination of slant insert 10 and slant base 20. As illustrated, an exemplary T-shaped stem 4 of an exemplary slant insert 10 may be engaged in T-shaped cavity 24b to hold slant insert 10 within the slant base 20. As may be seen in FIGS. 6-7, the joining of an exemplary slant insert 10 and slant base 20 may result in substantial alignment of each of their own non-orthogonal faces 1 and 21b, respectively. Alternatively, as may be illustrated in FIG. 8, an exemplar slant insert 10 may have its non-orthogonal face 1 misaligned with the non-orthogonal face 21b of slant base 20.

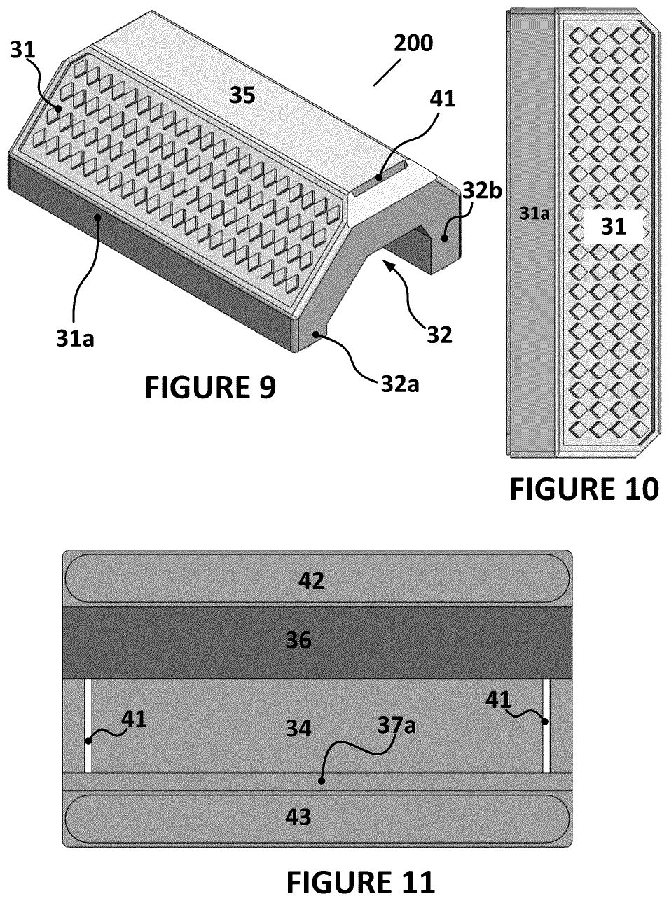

Referring to FIGS. 9-13, an ankle block 200 may be illustrated having at least non-orthogonal face 31, a top face 35, a thickness 32, a front foot component 32a, and a rear foot component 32b that may be lined with a friction-enhancing surface 42 and 43, respectively. In an exemplary embodiment, friction-enhancing surfaces 42, 43 may be non-slip rubber materials or treads. In an exemplary embodiment, ankle block 200 may be a unitary structure formed by injection molding, welding, bending, additive manufacturing, and other processes known to those skilled in the manufacturing arts. In another exemplary embodiment, ankle block 200 may have a textured surface on face 31 to increase friction or promote particular types of interactions with the end user. An exemplary ankle block 200 may have an interior cavity made of surfaces 36, 37a, and 37b for purposes of stacking similar ankle block 200 atop on another ankle block 200 and/or increasing stability of use of ankle block 200 by a user or facility of storage of the same. Additionally, an exemplary ankle block 200 may have channels 41 through its thickness 32 from the top face 35 to another interior cavity surface, e.g., face 34, 36, 37a-b. In a preferred embodiment, the channel 41 extends from the top face 35 to the face 34 for attachment of straps or other forms of portable transfer of the ankle block 200. Alternatively, channel 41 may be used to connect multiple ankle blocks 200 to one another to extend the ankle block 200 over a greater length. In this way, an exemplary ankle block 200 may present the added benefit of modularity in stretch systems. An exemplary connection for multiple ankle blocks 200 may be a suitably formed C-shaped bracket that slips into the corresponding channel 41 of each adjacent block 200. While channel 41 may be illustrated, those skilled in the art may contemplate numerous other mechanical fastening mechanisms that may be used on an exemplary ankle block 200, such as, for example, lanyards, carabiners, hook and loop fasteners, snap-fit, buckles, buttons, rope/string/wire.

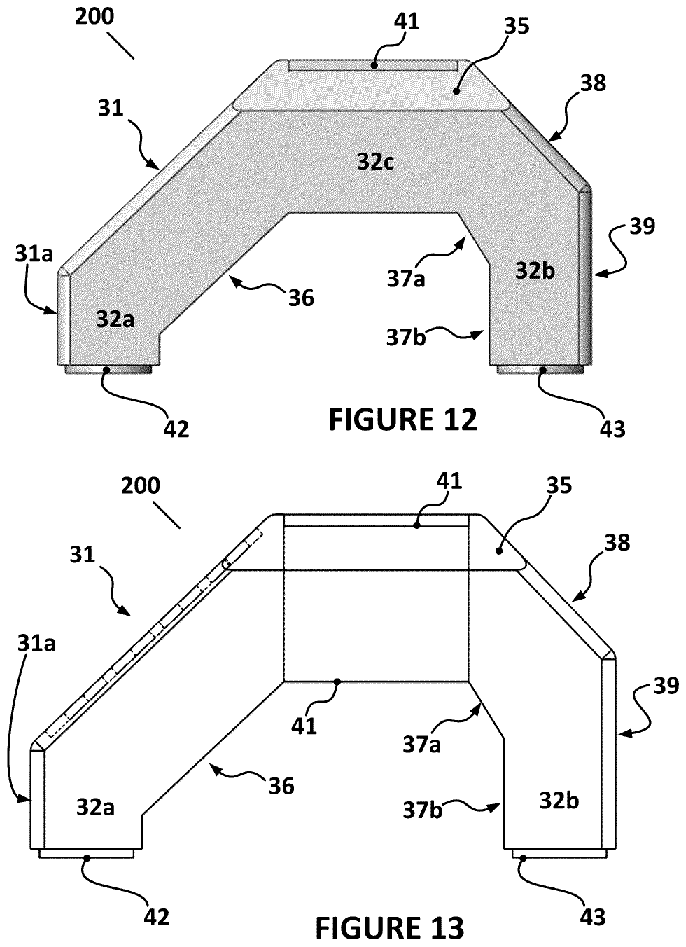

With continued reference to FIGS. 12 and 13, an exemplary ankle block 200 may have an intermediate thickness 32c that differs from either of the front foot 32a or the rear foot 32b. According to this illustrative embodiment, faces 37a and 37b are interconnected to rear faces 38 and 39 of the ankle block 200 via thicknesses 32b and 32c. Thus, an exemplary ankle block 200 may have numerous non-uniform and asymmetrical cross-sections depending on needs. Further, as illustrated in FIGS. 12-13 and 16-17, exemplary ankle block 200 may have two non-orthogonal surfaces located adjacent top face 35: face 31 and face 38. In an exemplary embodiment, the angle between face 31 and the support surface S is less than the angle between face 38 and the support surface S.

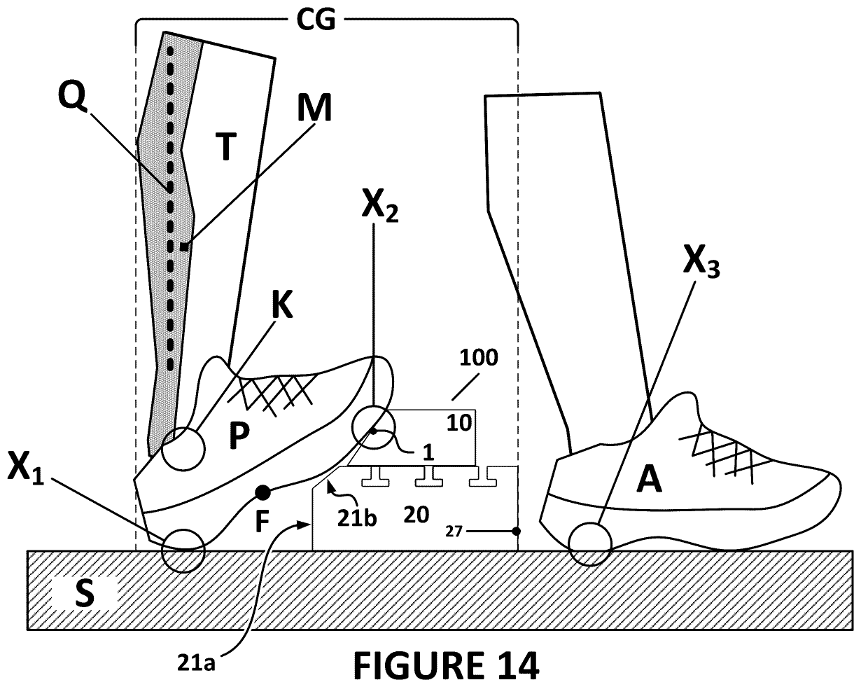

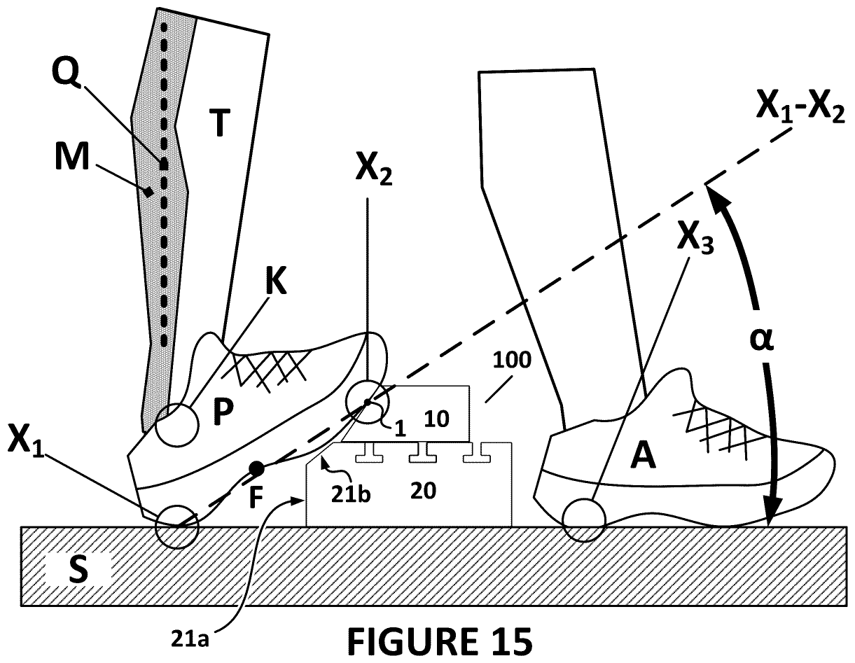

FIGS. 14-15 may illustrate an exemplary use case for an exemplary slant insert 10 and slant base 20 by a human user with legs configured according to the Normal Gait Cycle, preferably the stance phase and/or the swing phase, and most preferably, a sub-phase of the stance phase (e.g., the mid-stance phase). As illustratively provided, the posterior foot P of an exemplary user may be placed into dorsiflexion by assuming a position in which the posterior-most portion of the foot P (e.g., the heel) is in contact with the support surface S at point X1 and a portion of the sole of the foot P contacts face 1 of slant insert 10 at point X2. As illustrated, a flexion is induced in the ankle region K of the posterior foot P. The posterior-most portion of the anterior foot A is in contact with support surface S at point X3. In an exemplary embodiment, point X3 is such that anterior foot A is distal from either of slant insert 10 or slant base 20. In another exemplary embodiment, point X3 is such that anterior foot A is distal only from slant insert 10 but is otherwise in contact with slant base 10. In yet another embodiment, anterior foot A may be configured so that it is substantially orthogonal with respect to the direction of posterior foot P (e.g., rotation the stride of anterior foot A vis-à-vis posterior foot P).

As may be illustratively provided for in FIGS. 14-15, an exemplary user with posterior foot P positioned at points X1 and X2 may induce stretching in a muscle region M. In an exemplary embodiment, muscle region M may comprise one or more of the following constituents: gastrocnemius lateral, gastrocnemius medial, plantaris, tibiallis posterior, flexor digitorum longus, flexor hallucis longus, peroneus longus, peroneus brevis, soleus, and the Achilles tendon. In an exemplary embodiment, one or more constituents of the muscle region M may be stretched and while stretching contact a straight line Q that is perpendicular to the support surface S. According to a number of exemplary embodiments, line Q may be one or more of the following: a line that passes through the center of gravity (“CG”) of the user, a line that is posterior of face 21b, a line that is posterior of face 27. In another exemplary embodiment, a line that is perpendicular to support surface S and which passes through the CG of the user may be either: between line Q and face 21b, between face 21b and face 7, between face 7 and face 27, or between face 27 and point X3.

In an exemplary embodiment where the heel of posterior foot P and the posterior foot P's non-orthogonal face-contacting portion are each positioned at points X1 and X2, respectively, one or more constituents of the muscle region M is stretched while being in contact with straight line Q. Accordingly, an exemplary straight line Q may be one or more of the following: a line that passes through the center of gravity (“CG”) of the user, a line that is posterior of face 21b, a line that is posterior of face 27. In an exemplary embodiment where the heel of posterior foot P and the posterior foot P′s non-orthogonal face-contacting portion are each positioned at points X1 and X2, respectively, one or more constituents of the muscle region M is stretched and a line that is perpendicular to support surface S and which passes through the CG of the user may be either: between line Q and face 21b, between face 21b and face 7, between face 7 and face 27, or between face 27 and point X3.

In a still further exemplary embodiment where the heel of posterior foot P and the posterior foot P's non-orthogonal face-contacting portion are each positioned at points X1 and X2, respectively, one or more constituents of the muscle region M is stretched while a line X₁-X₂ passes through points X1 and X2 at an angle α that is within the range of 25° to 70° with respect to the support surface S, preferably a range between 35° and 60°, more preferably between 40° and 50° with respect to the support surface S, and more preferably 45.0°.

In an exemplary embodiment where the heel of posterior foot P and the posterior foot P's non-orthogonal face-contacting portion are each positioned at points X1 and X2, respectively, one or more constituents of the muscle region M is stretched and a line that is perpendicular to support surface S and which passes through the CG of the user may be either: between line Q and face 21b, between face 21b and face 7, between face 7 and face 27, or between face 27 and point X3. Further according to this particular exemplary embodiment, one or more constituents of the muscle region M is stretched while a line X₁-X₂ passes through points X1 and X2 at an angle α that is within the range of 25° to 70° with respect to the support surface S, preferably a range between 35° and 60°, more preferably between 40° and 50° with respect to the support surface S, and more preferably 45.0° with respect to the support surface S.

In one or more of the foregoing embodiments illustratively provided for in FIGS. 14-17, adjusting the CG allows the user to engage the plantar fascia at point F. Additionally and/or alternatively, the talocural joint may also be mobilized as the tibia T will forward on the talus of the user due to the anterior foot A being disposed forward of the posterior foot P.

An exemplary ankle block system 100 may induce a stretch in any combination of gastrocnemius lateral, gastrocnemius medial, plantaris, tibiallis posterior, flexor digitorum longus, flexor hallucis pongus, peroneous longus, peroneus brevis, soleus, and the Achilles tendon or may only induce a stretch in those constituents of muscle group M that are most proximal to ankle block 100. Alternatively, an exemplary ankle block system 100 may induce a stretch in any combination of gastrocnemius lateral, gastrocnemius medial, plantaris, tibiallis posterior, flexor digitorum longus, flexor hallucis pongus, peroneous longus, peroneus brevis, soleus, and the Achilles tendon or may only induce a stretch in those constituents of muscle group M that are most distal to ankle block 100.

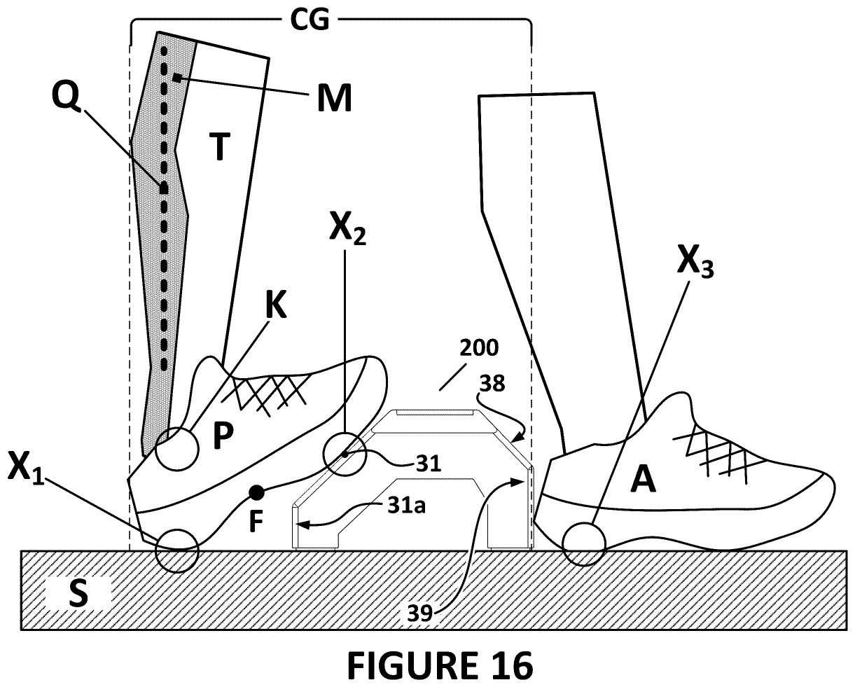

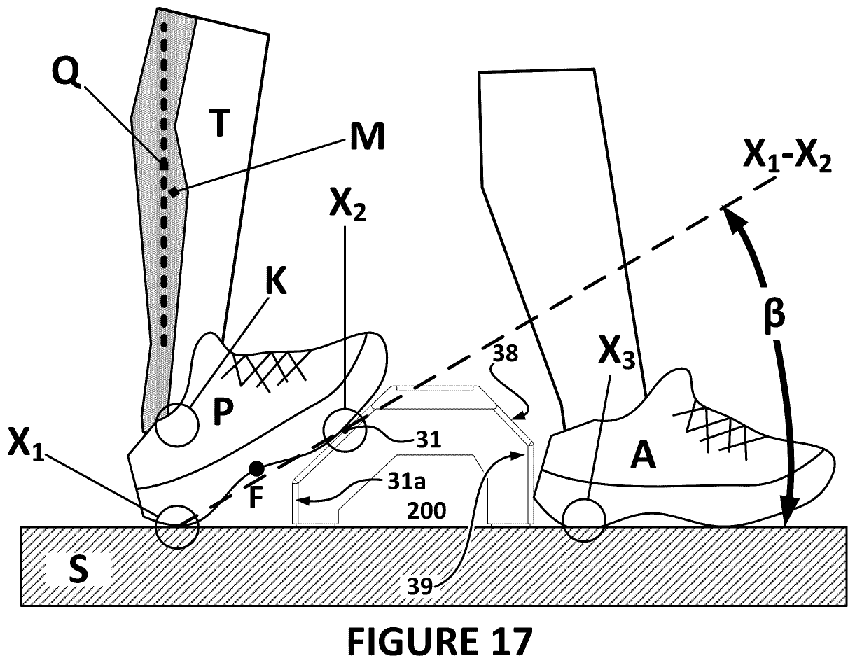

FIGS. 16-17 may illustrate an exemplary use case for an exemplary ankle block 200 by a human user with legs configured according to the Normal Gait Cycle, preferably the stance phase and/or the swing phase, and most preferably, a sub-phase of the stance phase (e.g., the mid-stance phase). As illustratively provided, the posterior foot P of an exemplary user may be placed into dorsiflexion by assuming a position in which the posterior-most portion of the foot P (e.g., the heel) is in contact with the support surface S at point X1 and a portion of the sole of the foot P contacts face 31 of ankle block 200 at point X2. As illustrated, a flexion is induced in the ankle region K of the posterior foot P. The posterior-most portion of the anterior foot A is in contact with support surface S at point X3. In an exemplary embodiment, point X3 is such that anterior foot A is distal from either face 39 or face 38 of ankle block 200. In another exemplary embodiment, point X3 is such that anterior foot A is distal only from face 38 but is otherwise in contact with ankle block 200 via face 39. In yet another embodiment, anterior foot A may be configured so that it is substantially orthogonal with respect to the direction of posterior foot P (e.g., rotation the stride of anterior foot A vis-à-vis posterior foot P).

As may be illustratively provided for in FIGS. 16-17, an exemplary user with posterior foot P positioned at points X1 and X2 may induce stretching in a muscle region M. In an exemplary embodiment, muscle region M may comprise one or more of the following constituents: gastrocnemius lateral, gastrocnemius medial, plantaris, tibiallis posterior, flexor digitorum longus, flexor hallucis pongus, peroncous longus, peroneus brevis, soleus, and the Achilles tendon. In an exemplary embodiment, one or more constituents of the muscle region M may be stretched and while stretching contact a straight line Q that is perpendicular to the support surface S. According to a number of exemplary embodiments, line Q may be one or more of the following: a line that passes through the CG of the user, a line that is posterior of face 31a, a line that is posterior of face 38, or a line that is posterior of face 39. In another exemplary embodiment, a line that is perpendicular to support surface S and which passes through the CG of the user may be either: between line Q and face 31a, between face 31a and face 38, between face 38 and face 39, or between face 39 and point X3.

In an exemplary embodiment where the heel of posterior foot P and the posterior foot P's non-orthogonal face-contacting portion are each positioned at points X1 and X2, respectively, one or more constituents of the muscle region M is stretched while being in contact with straight line Q. Accordingly, an exemplary straight line Q may be one or more of the following: a line that passes through the center of gravity (“CG”) of the user, a line that is posterior of face 31a, a line that is posterior of face 38, or a line that is posterior of face 39. In an exemplary embodiment where the heel of posterior foot P and the posterior foot P's non-orthogonal face-contacting portion are each positioned at points X1 and X2, respectively, one or more constituents of the muscle region M is stretched and a line that is perpendicular to support surface S and which passes through the CG of the user may be either: between line Q and face 31a, between face 31a and face 38, between face 38 and face 39, or between face 39 and point X3.

In a still further exemplary embodiment where the heel of posterior foot P and the posterior foot P's non-orthogonal face-contacting portion are each positioned at points X1 and X2, respectively, one or more constituents of the muscle region M is stretched while a line X₁-X₂ passes through points X1 and X2 at an angle β that is within the range of 25° to 80° with respect to the support surface S, preferably a range between 40° and 65°, more preferably between 42° and 55° with respect to the support surface S, and more preferably 45.0° with respect to the support surface S.

In an exemplary embodiment where the heel of posterior foot P and the posterior foot P's non-orthogonal face-contacting portion are each positioned at points X1 and X2, respectively, one or more constituents of the muscle region M is stretched and a line that is perpendicular to support surface S and which passes through the CG of the user may be either: : between line Q and face 31a, between face 31a and face 38, between face 38 and face 39, or between face 39 and point X3. Further according to this particular exemplary embodiment, one or more constituents of the muscle region M is stretched while a line X₁-X₂ passes through points X1 and X2 at an angle β that is within the range of 25° to 80° with respect to the support surface S, preferably a range between 40° and 65°, more preferably between 42° and 55° with respect to the support surface S, and more preferably 45.0° with respect to the support surface S.

An exemplary ankle block 200 may induce a stretch in any combination of gastrocnemius lateral, gastrocnemius medial, plantaris, tibiallis posterior, flexor digitorum longus, flexor hallucis pongus, peroneous longus, peroneus brevis, soleus, and the Achilles tendon or may only induce a stretch in those constituents of muscle group M that are most proximal to ankle block 200. Alternatively, an exemplary ankle block system 200 may induce a stretch in any combination of gastrocnemius lateral, gastrocnemius medial, plantaris, tibiallis posterior, flexor digitorum longus, flexor hallucis pongus, peroncous longus, peroneus brevis, soleus, and the Achilles tendon or may only induce a stretch in those constituents of muscle group M that are most distal to ankle block 200.

One exemplary advantage from the teachings herein is to provide a device that transfers the maximum amount of force from the user to the area of stretch. Another exemplary advantage is that the forces undertaken to use the device are in the same or substantially the same direction and reduce the need to balance against or combat the same during the stretching exercise. A still further exemplary advantage of the slant design described is that it transfers maximum weight in any direction. A still further exemplary advantage of the ankle block 200 or ankle block system 100 includes light weight design, portability, and use on multiple terrain and in multiple indoor and outdoor arrangements. Another exemplary advantage from the teachings herein is that the ankle block 200 allows the user to adjust different levels of leg muscle dorsiflexion without having to change the height of the product.

Another exemplary advantage of the teachings herein is that a user can assume various stances within the normal gait cycle while achieving dorsiflexion without any loss of stability. While reference has been made to alignment of a line Q with a perpendicular line connecting the CG to the support surface S, those skilled in the art may also use as a proxy for practice of the teachings herein the location of the greater trochanter of the femur and space on surface S between points X₁ and X₂.

Many further variations and modifications may suggest themselves to those skilled in art upon making reference to above disclosure and foregoing interrelated and interchangeable illustrative embodiments, which are given by way of example only, and are not intended to limit the scope and spirit of the interrelated embodiments of the invention described herein.

1. A muscle flexion device, comprising:
a polyhedron having a trapezoidal cross-section having a slanted face that is angled with respect to a support surface and a face perpendicular to the support surface; and
at least one contact surface on the slanted face, wherein
the polyhedron is configured to induce dorsiflexion in a posterior leg of a human user when the human user is positioned in the normal gait cycle whereby a posterior foot connected to the posterior leg simultaneously touches the at least one contact surface and the support surface, and
wherein an anterior leg touches the support surface forward of the face perpendicular to the support surface.

2. The muscle flexion device of claim 1, wherein the polyhedron has a unitary trapezoidal cross-section.

3. The muscle flexion device of claim 1, wherein the angle of the slanted face with respect to the support surface is 40° to 48°.

4. The muscle flexion device of claim 1, wherein the polyhedron is configured to place a center of gravity of the human user over at least one muscle in the posterior leg.

5. The muscle flexion device of claim 1, wherein the polyhedron is configured to place a center of gravity of the human user over at least one portion of the polyhedron.

6. The muscle flexion device of claim 1, wherein the polyhedron is a trapezoid.

7. The muscle flexion device of claim 6, wherein the polyhedron is a trapezoid and the angle of the slanted face with respect to the support surface is 40° to 48°.

8. The muscle flexion device of claim 1, wherein a portion of the polyhedron comprising the slanted face is removable from the rest of the device.

9. The muscle flexion device of claim 1, wherein the anterior leg does not touch the polyhedron.

10. A method of flexing a muscle in a posterior leg of a user positioned in a normal gait cycle, comprising the steps of:
contacting with a foot of the posterior leg a support surface and a slanted surface of a polyhedral block having a trapezoidal cross-section and resting on the support surface; and
placing the foot of the anterior leg forward of the polyhedral block with the trapezoidal cross-section.

11. The method of claim 10, further comprising the step of positioning a center of gravity of the user over at least one muscle group in the posterior leg.

12. The method of claim 10, wherein the foot of the posterior leg when in contact with the polyhedral block is angled at approximately 25° to 80° with respect to the support surface.

13. The method of claim 10, wherein the foot of the posterior leg when in contact with the polyhedral block is angled at approximately 35° and 60° with respect to the support surface.

14. The method of claim 10, wherein the foot of the posterior leg when in contact with the polyhedral block is angled at approximately 40° and 50° with respect to the support surface.

15. The method of claim 10, further comprising the step of positioning a center of gravity of the user forward of a line contacting at least one stretched muscle group in the posterior leg.

16. The method of claim 10, further comprising the step of coupling the slanted surface portion of the polyhedral block to a remainder portion of the polyhedral block.

17. The method of claim 10, further comprising placing the foot of the anterior leg in contact with the polyhedral block.

18. A polyhedral block configured to induce dorsiflexion in a posterior leg of a human user upon contact with a posterior foot of the posterior leg of the same while the human user is in a Normal Gait cycle and the posterior foot is simultaneously in touch with a support surface and at least one slanted surface on the polyhedral block, the polyhedral block comprising:
at least one flat surface in contact with a support surface; and
at least one side surface opposite the at least one slanted surface, wherein the at least one side surface, the at least one flat surface, and the at least one slanted surface are surfaces of a trapezoidal cross-section.

19. The polyhedral block of claim 18, wherein the at least one slanted surface is at an angle between approximately 25° to 80°.

20. The polyhedral block of claim 18, further comprising another slanted surface disposed on the same side as the at least one side surface.
